(19) United States
(12) Plant Patent Application Publication
(10) Pub. No.: US 2013/0291247 P1
(43) Pub. Date: Oct. 31, 2013

(54) Phalaenopsis orchid plant named 'Lions Dive'

(50) ;

(71) Applicant: FLORICULTURA, Heemskerk (NL)

(72) Inventor: René Schoone, Assendelft (NL)

(21) Appl. No.: 13/986,290

(22) Filed: Apr. 19, 2013

Related U.S. Application Data
(60) Provisional application No. 61/638,441, filed on Apr. 25, 2012.

(51) Int. Cl. A01H 5/00

(52) U.S. Cl. PLT/311

(57) ABSTRACT

A new and distinct Phalaenopsis plant named ‘Lions Dive’ particularly characterized by flowers which are purple/violet with white edges; plants which may be propagated economically and uniformly using tissue culture; plants which produce more than one inflorescence; long and sturdy inflorescences; and relatively short, dark-green foliage.

### LATIN NAME OF THE GENUS AND SPECIES OF THE PLANT CLAIMED

Phalaenopsis hybrid

### VARIETY DENOMINATION

‘Lions Dive’

### BACKGROUND OF THE INVENTION

The present invention relates to a new and distinct cultivar of Phalaenopsis plant, botanically known as Phalaenopsis of the Orchidaceae family, and hereinafter referred to by the cultivar name ‘Lions Dive’.

Phalaenopsis comprises a genus of about 55 species of herbaceous perennials many of which, or the hybrids thereof, are suitable for cultivation in the home or greenhouse. Phalaenopsis is predominantly epiphytic or rock-dwelling, and is native to tropical Asia, the Malay Archipelago, and Oceania. The species typically has 2-ranked, fleshy, oblong or elliptic leaves affixed to a short central stem (monopodial growth), which vary in size from 5 to 8 inches to over 2 feet. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes, often with many showy flowers which open in succession beginning with the lowermost. The flowers possess three sepals and three petals; the lateral ones being alike. The lowermost petal, called the labellum, is three-lobed and is often more brightly-colored than the other flower segments. Flower colors include various shades of pink, white, yellow and red-brown.

Phalaenopsis orchids are typically propagated from seeds. Asexual propagation of Phalaenopsis is often done from off-shoots which frequently arise from the lower bracts of the inflorescence. The resulting plants are detached from the mother plant and may be planted in a suitable substrate.

The new Phalaenopsis ‘Lions Dive’ is a product of a controlled breeding program conducted by the inventor, René Schoone, in Strengweg, Heemskerk, The Netherlands. The objective of the breeding program was to develop a new Phalaenopsis cultivar particularly characterized by its attractive and unique colored flowers, economical propagation via tissue culture, rapid growth, and a plant dimension suitable for packaging and shipping to the market.

The new Phalaenopsis ‘Lions Dive’ originated from a cross made by the inventor in 1999 in Strengweg, Heemskerk, The Netherlands. The female or seed parent is the Phalaenopsis cultivar designated ‘Little Mary×Pico Caroline’, unpatented. The male or pollen parent is the Phalaenopsis cultivar designated ‘Pico Sweetheart’, unpatented. The new Phalaenopsis ‘Lions Dive’ was discovered and selected by the inventor as a single flowering plant within the progeny of the stated cross in a controlled environment in 2007 in Strengweg, Heemskerk, The Netherlands.

Asexual reproduction of the new Phalaenopsis cultivar by tissue culture was first performed in November, 2007 in Cieweg 13, Heemskerk, The Netherlands, and has demonstrated that the combination of characteristics as herein disclosed for the new cultivar are firmly fixed and retained through successive generations of asexual reproduction. The new cultivar reproduces true to type.

### BRIEF DESCRIPTION OF THE INVENTION

The following traits have been repeatedly observed and are determined to be unique characteristics of ‘Lions Dive’, which in combination distinguish this Phalaenopsis as a new and distinct cultivar:
1. flowers which are purple/violet with white edges;
2. plant produces more than one inflorescence;
3. plants may be propagated economically and uniformly using tissue culture;
4. inflorescences are long and sturdy; and
5. relatively short, dark-green foliage.
   In comparison with the parental cultivars of ‘Lions Dive’, the female parent ‘Little Mary×Pico Caroline’ has dark pink colored flowers and the male parent ‘Pico Sweetheart’ has pink colored flowers, whereas the flowers of ‘Lions Dive’ are purple/violet with white edges.
   Presently, there is no other commercial cultivar to which ‘Lions Dive’ can be meaningfully compared.

### BRIEF DESCRIPTION OF THE PHOTOGRAPHS

The accompanying photographs illustrate the overall appearance of the new Phalaenopsis ‘Lions Dive’ showing the colors as true as is reasonably possible with colored reproductions of this type. Colors in the photographs may differ slightly from the color values cited in the detailed botanical description, which accurately describe the color of ‘Lions Dive’.

FIG. 1 shows a side view perspective of a typical flowering plant of ‘Lions Dive’ in a 12 cm pot, at 16 months of age.

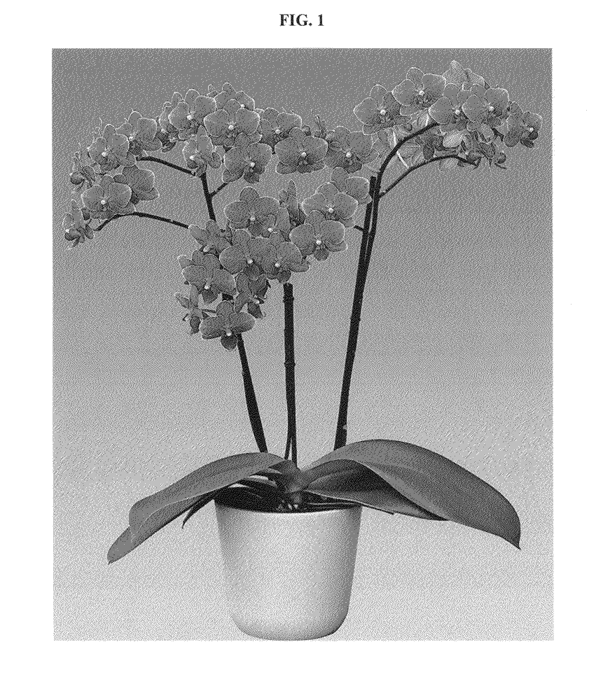

FIG. 2 shows a close-up view of the typical buds and flowers of ‘Lions Dive’.

FIG. 3 shows a close-up view of the typical leaves of ‘Lions Dive’.

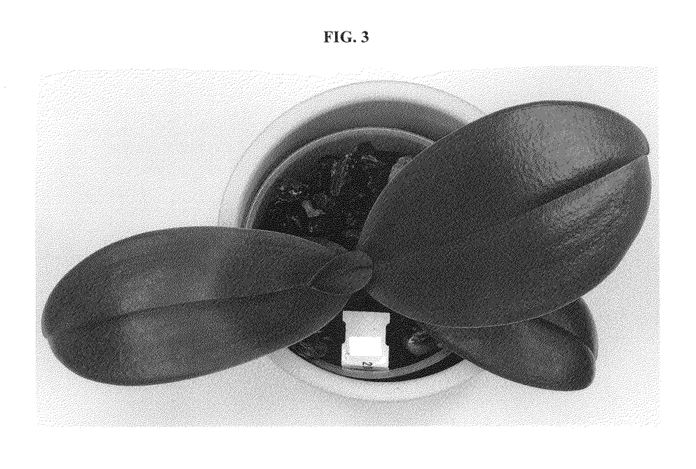

### DETAILED BOTANICAL DESCRIPTION

The new Phalaenopsis cultivar ‘Lions Dive’ has not been observed under all possible environmental conditions. The phenotype of the new cultivar may vary with variations in environment such as temperature, light intensity, and day length without any change in the genotype of the plant.

The aforementioned photographs, together with the following observations, measurements and values describe plants of ‘Lions Dive’ as grown in a greenhouse in Strengweg, Heemskerk, The Netherlands, under conditions which closely approximate those generally used in commercial practice. Initially, the ideal temperature to grow plants of ‘Lions Dive’ is 27° C. during the day and at night. Then, during the flowering phase of ‘Lions Dive’, the ideal growing temperature is 20-22° C. during the day and 18° C. at night. Light levels for growing ‘Lions Dive’ are a minimum of 5,000 lux and a maximum of 10,000 lux. A balanced fertilizer with level of 200 ppm N, 87 ppm P, 168 ppm K is applied. Duration of growth of ‘Lions Dive’ from potting size is between 10 and 14 months.

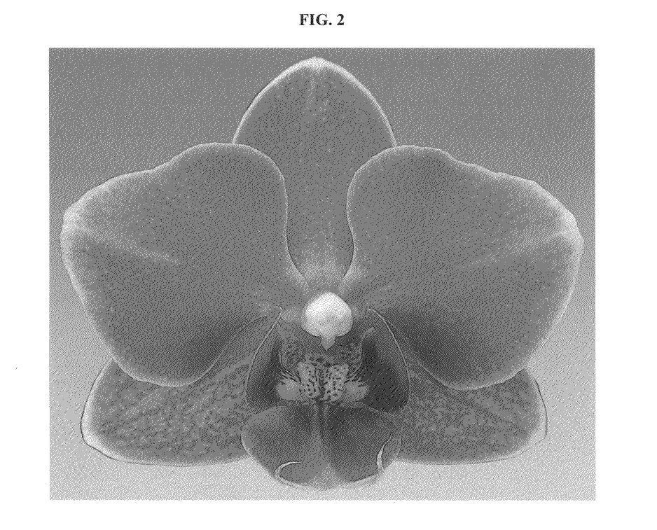

Color references are made to The Royal Horticultural Society Colour Chart (R.H.S.), 2007 edition, except where general colors of ordinary significance are used. Color values were taken under daylight conditions at approximately noon in Zaandammerweg, Assendelft, The Netherlands. The age of the ‘Lions Dive’ plants described is 12 months after potting.
Classification:
   Botanical.—Phalaenopsis hybrid.
Parentage:
   Female or seed parent.—Phalaenopsis cultivar designated ‘Little Mary×Pico Caroline’, unpatented.
   Male or pollen parent.—Phalaenopsis cultivar designated ‘Pico Sweetheart’, unpatented.
Propagation:
   Type.—tissue culture.
   Rooting habit and description.—Fleshy; approximately 4 mm-9 mm wide and green in color (RHS 146B); freely branching. It takes 12 weeks for plants growing in tissue culture to initiate roots.
Plant:
   Size at maturity.—Height: about 52 cm Spread: about 45 cm.
   Growth habit.—small; dark green leaves and a relatively normal raceme.
   Vigor.—moderate.
   Crop time.—Following asexual propagation, at about 26 weeks 2 leaves appear; at about 30 weeks 3-4 leaves appear; after a cold treatment of about 4-8 weeks at a temperature of about 19° C. about 2 peduncles with flowers appear.
Foliage:
   Quantity per plant.—About 6 to 8 leaves are produced before flowering.
   Arrangement and attachment.—half up/horizontal and on two sides.
   Overall shape of leaf.—oval, the tip is blunt and asymmetric.
   Texture (upper & underside).—smooth and leathery.
   Pubescence.—3 to 4 pairs of leaves.
   Mature leaf length.—about 14 to 20 cm.
   Mature leaf width.—about 6 to 10 mm.
   Mature leaf thickness.—about 2 mm.
   Mature leaf color.—upper side: green (RHS 137A) Under side: yellow/green (RHS 146B).
   Leaf base.—acute.
   Margin.—entire.
   Venation.—Pattern: parallel Color of midvein: upper side: green (RHS N137A) under side: yellow/green (RHS 144A).
Raceme:
   Quantity per plant.—about 1 to 3.
   Number of flowers per raceme.—about 15 to 30.
   Length.—about 45 to 50 cm.
   Diameter.—about 4 mm.
   Strength.—strong.
   Aspect.—upright.
   Texture.—glabrous and smooth.
   Color.—yellow/green (RHS 147A) mixed with yellow/green (RHS 144A).
   Internode.—Length: about 24 to 32 mm.
Inflorescence description:
Appearance: Upright to slightly pendant, racemose inflorescence with bilaterally symmetrical flowers that open in succession beginning with the lowermost flower.
Buds:
   Height (from base to tip).—about 12 mm to 15 mm.
   Diameter (at midpoint).—about 7 mm to 15 mm.
   Shape.—egg-shaped/oval.
   Color.—yellow/green (RHS 144B) and some purple (RHS N79C).
Flowering time: For an untreated plant (flowering plant that has not undergone cold-treatment where the plant grows at a temperature of 18° C. to 19° C. for about 4 to 8 weeks after a period of about 30 weeks at a temperature of 25° C.), 2 racemes appear with about 16 to 20 flower buds and flowers per inflorescence. First flowers can be expected approximately 4 to 6 months after planting a plant with a leaf diameter of 3 to 5 cm. Flowers persistent.
Flowering longevity: On the plant: about 4 to 6 months; lastingness of cut flowers: has not been observed
Fragrance: No fragrance
Flower:
   Rate of opening.—Flowers fully opened about 2 to 3 days after petal and sepal separation.
   Orientation at opening.—slanted upward and outward.
   Shape.—see FIG. 2.
   Size.—Height: about 35 mm to 50 mm Diameter: about 44 mm to 50 mm Depth of tube: about 8 mm.
Petals:
   Quantity and arrangement.—six petals that are trimerous, overlapping and arranged in 2 whorls. Petals are more pronounced than sepals.
   Arrangement.—Inner whorl of petals comprises 3 petals, 2 lateral petals and labellum.
   2 lateral petals.—Overall shape: little triangular Apex: oval Margin: entire and weakly undulate Base: broadly ovate Length: about 25 mm Width: about 23 mm Texture: Upper surface: smooth and satiny Under surface: smooth and satiny Color (when fully opened): upper side: Main color is purple/violet (RHS N81B) with white edges (RHS NN155C). Under side: Main color is purple/violet (RHS N81). Edges are white (RHS NN15 C) and in the center a white haze (RHS NN155C). Labellum: Overall shape: 3-lobed with 2 prominent callosities at central junction of the lateral lobes and base of the midlobe. Lateral lobes of labellum fold upward about the column; the midlobe extends forward and is terminated by 2 short filiform appendages at the apex. Lateral lobes of the labellum are ovate in shape while the midlobe is triangular with a bump and a rib on it Margin: entire Apex: oval Length: about 18 mm Width (not flattened): about 15 mm Texture: Upper and under surface: smooth and satiny Color (when fully opened): Midlobe: upper surface: At the base some grey/orange (RHS 167A) which runs into purple/violet (RHS N80A). From halfway to end white edges (RHS NN155C). Under surface: At the base left and right small edge of grey/orange (RHS 167A), which runs into a haze of purple/violet (RHS N82A). In the center purple (RHS 76C). Lateral lobes: upper surface: At the base white (RHS NN155C) with a yellow spot (RHS 13A). Lower edge is grey/orange (RHS 167A). Close to base small red/purple stripes (RHS 61A). Than an edge RHS 60A, which runs into purple/violet (RHS N80A). Under surface: At the base white (RHS NN155C) which runs into purple (RHS 77A) and after that RHS N80A, with at the lower edge some grey/orange (RHS 167A) Chirri: short (about 5 mm) color: purple/violet (RHS N81C) with white tips (RHS NN155C) Pestle (Callosities): Length: about 4 mm Width (not flattened): about 4 mm Color: Sides are white (RHS NN155C). Front, the edges and inside yellow (RHS 13A). Spots are red/purple (RHS 59A).
Sepals:
   Arrangement.—Outer whorl comprises 3 sepals.
   Overall shape.—oval and narrow Margin: entire and weakly undulate Length: about 24 mm Width: about 15 mm Apex: oval/round, little bit pointed Texture: Upper and under surface: smooth and satiny Color (when fully opened): upper side: Main color is purple/violet (RHS N81B) with white edges (RHS NN155C). At the base of the lateral sepals a white mark (RHS N155D/154D) with red/purples spots (RHS 59A). Under side: main color is purple (RHS 74C) with white edges (RHS NN155C). At the base of the lateral sepals a small mark (RHS 154C).
Reproductive organs:
Arrangement: The stamens, style and stigmas are fused into a single, short structure called the column, possessing one terminal anther with pollen grains united into a pollinia, which are covered by an anther cap. The stigma is located under the column behind the pollinia. The ovary is inferior with three carpels present. The plant has not produced seed.
Column:
   Length.—about 10 mm.
   Diameter.—about 6 mm.
   Color.—Purple/violet (RHS N81C) and white (RHS NN155C).
   Pollinia.—.
   Quantity.—two.
   Diameter.—about 1 mm.
   Color.—orange (RHS 25A).
Ovary:
   Length.—about 3 mm.
   Diameter.—about 4 mm.
   Color.—white (RHS NN155C) and purple edge (RHS N81C).
Pedicel:
   Length.—about 32 mm.
   Diameter.—about 3 mm.
   Texture.—glabrous and smooth.
   Color.—from yellow/green (RHS 147B) to white (RHS N155B).
Disease/pest resistance/susceptibility: No specific resistance or susceptibility observed
Temperature tolerance: Tolerant to a low temperature of about 15° C. and to a high temperature about 30° C.

What is claimed is:

1. A new and distinct Phalaenopsis plant named ‘Lions Dive’, as illustrated and described herein.
